(19) United States
(12) Patent Application Publication
(10) Pub. No.: US 2005/0009116 A1
(43) Pub. Date: Jan. 13, 2005

(54) Reagents, methods and kit for detecting feed enzymes

(75) Inventors: Michele Susan Yarnell, Research Triangle Park, NC (US); Lilian Zeitouni, Research Triangle Park, NC (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Participation AG

(21) Appl. No.: 10/884,560

(22) Filed: Jul. 2, 2004

Related U.S. Application Data
(60) Provisional application No. 60/485,602, filed on Jul. 7, 2003.

(51) Int. Cl. G01N 33/53

(52) U.S. Cl. 435/7.9

(57) ABSTRACT

This invention relates to the field of immunology and more specifically relates to immunoassay methods, kits, and reagents, for the detection of proteins and enzymes, in particular feed enzymes.

This application claims the benefit of the filing date of U.S. Provisional Patent Application No. 60/485,602 filed Jul. 7, 2003, which is hereby incorporated by reference in its entirety.

### FIELD OF THE INVENTION

This invention relates to the field of immunology and more specifically relates to immunoassay methods, including ELISA and immunostrip assays, kits and reagents, for the detection of proteins and enzymes, in particular feed enzymes.

### BACKGROUND OF INVENTION

There is a significant need for a convenient, relatively easy assay for detecting the presence of enzymes in animal feed. To date, there is no standard immunological assay for such detection.

There is also a tremendous need for determining whether a plant has been genetically modified or whether grain or processed foods contain GMO traits. The need requires test methods that can detect and quantitate either the novel DNA or protein. The present inventions meet this need by providing immunological methods, reagents and kits for detection and quantification of feed enzymes.

### SUMMARY OF THE INVENTION

Methods, kits, and reagents for detecting and measuring an enzyme or enzymes in a sample are provided. Preferably, the proteins to be detected include, but are not limited to one or more phytases, xylanases, cellulases, glucanases, amylases, glucoamylases, and proteases. The proteins may be produced in various micro-organisms, including but not limited to Esherichia coli, Schizosaccharomyces pombe, and Pichia pastoris or in plants, including but not limited to maize, wheat, rice, canola, and alfalfa, for example. In particular, the proteins are detected in feed or in genetically modified plants containing a gene encoding the protein. The feed is animal feed. The animal feed may be for monogastrics or ruminants. The feed may be mash feed and/or pelleted feed.

The reagents include purified protein and antibodies specific for the protein. In a preferred embodiment, the protein is a phytase. The phytase protein may be isolated from E. coli inclusion bodies and administered to animals to produce polyclonal or monoclonal antibodies. Alternatively, the protein may be isolated from a soluble cell extract, such as an E. coli cell extract.

The antibodies have high sensitivity and specificity for the protein and are useful in immunoassay methods for the detection of enzymatically active protein in animal or in genetically modified organisms.

The methods are immunoassays employing antibodies described herein and are capable of detecting low concentrations of protein. The antibodies are purified and therefore react minimally with other proteins that may be present in the sample.

The antibodies and/or protein are assembled in a kit with conventional immunoassay reagents for detection of the protein.

A method, kit and reagents for detecting and measuring protein in a sample are provided. The proteins to be detected include one or more feed enzymes, such as phytases, xylanases, cellulases, glucanases, amylases, glucoamylases, and proteases. The proteins may be produced from various species, including but not limited to E. coli, S. pombe, and P. pastoris. In particular, the phytase proteins are detected in animal feed and in genetically modified plants expressing a desired feed enzyme gene, such as a phytase gene.

The reagents may include antigenic peptides/proteins and antibodies. The antigenic peptides/proteins are immuno-reactive with the antibodies. The antigenic peptides/proteins have common epitopes shared by the protein produced in different species. The peptides/proteins are isolated or synthesized and administered to animals to produce antibodies.

For phytase, the antibodies have high sensitivity and cross-reactivity for phytase proteins produced in various species and are therefore useful in immunoassay methods for the detection of genetically modified organisms, particularly plants, which have been engineered to express a phytase gene.

The methods are immunoassays employing antibodies described herein and are capable of detecting low concentrations of phytase protein in animal feed and genetically enhanced crop samples. The antibodies are immunoreactive with epitopes or common epitopes on phytase expressed by phytase genes and react minimally with other proteins that may be present in the sample, thus providing for an accurate determination of the presence of a genetically modified organism in a sample, such as a grain sample.

The epitopes, antibodies, or both, are collectively assembled in a kit with conventional immunoassay reagents for detection of protein. The kit may optionally contain both monoclonal and polyclonal antibodies and a standard for the determination of the presence of protein or feed enzyme in a sample.

In view of the above, there is a real need for the development of technology that will allow the identification of specific proteins or feed enzymes in samples.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a graph showing a standard curve for phytase activity.

FIG. 2 is a graph showing the percent relative activity versus incubation time at 99° C. of the phytase enzyme in both an ELISA and an enzyme-activity assay. The detection of phytase enzyme in the ELISA parallels the amount of activity detected in the enzyme-activity assay.

FIG. 3 is a scanned reproduction of immunostrip tests showing the detection of phytase (arrow) after incubation at 99° C. for up to one hour. A decrease in the detection of phytase is seen after about 20 minutes at 99° C.

FIG. 4 is a depiction of an exemplary immunoassay test kit and the method of using the same.

### DETAILED DESCRIPTION OF THE DISCLOSED EMBODIMENTS

Methods, kits, and reagents for the detection of proteins in a sample are described herein. Preferably, the proteins are feed enzymes, and more preferably the feed enzymes include, but are not limited to, phytases, xylanases, cellulases, glucanases, amylases, glucoamylases, and proteases.

The methodology of the invention may be used to detect any enzyme in samples such as animal feed. Many feed enzymes are known to those skilled in the art. For example, a number of phytases are known, the detection of which may be accomplished using the present invention. Known phytases include, but are not limited to, those described in WO 01/90333, entitled “Recombinant Bacterial Phytases and Uses Thereof;” WO 99/08539, entitled “Novel Phytase;” U.S. application Ser. No. 10/334,672, entitled “Microbially Expressed Thermotolerant Phytase For Animal Feed”, and U.S. application Ser. No. 10/334,671, entitled “Thermotolerant Phytase for Animal Feed,” each and all of which are incorporated by reference herein in their entirety.

It is important when making immunoassays to detect protein in transgenic plants and the products produced from them (including food fractions), that a test has the capacity to detect the protein from various genes. Thus, cross-reactive antibodies are very important for development of successful commercial products.

The reagents are antigenic protein or peptides sharing common epitopes and anti-protein antibodies that are cross-reactive with the protein expressed from different genes. The method is an immunoassay for the sensitive, specific detection of protein, specifically for the detection of protein in animal feed and in genetically engineered plants, such as agricultural products. The kit contains the anti-protein antibodies described herein and other reagents, particularly those used in a strip test format, for use in the immunoassay described in more detail below.

Antigenic Protein

For preparation of recombinant protein, such as phytase, following transformation of a suitable host strain and growth of the host strain to an appropriate cell density, e.g., a bacterial, insect or yeast host, a selected promoter may be induced by appropriate means (e.g., temperature shift or chemical induction) and cells cultured for an additional period to yield recombinant enzyme. Cells are then typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents, such methods are well known to those skilled in the art.

The enzyme can be recovered and purified from recombinant cell cultures by methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. Finally, high performance liquid chromatography (HPLC) can be employed for final purification steps.

Antigenic Peptides

Antigenic peptides may be protein surface peptides that share epitopes across various species expressing the protein, preferably protein expressed from various micro-organisms.

The peptides are highly useful as diagnostic markers for the detection and quantification of the protein. The peptides are also useful for producing antibodies, tests and kits having the superior sensitivity required of successful commercial products.

In one embodiment, the peptides are either isolated from cell cultures in which the protein-encoding genes are expressed using conventional techniques known to those skilled in the art such as affinity column purification or the amino acid sequences of the peptides are generated and the peptides synthesized in accordance with methods known to those in the art.

Antigenic peptides having the characteristics set forth above are useful for the production of either monoclonal or polyclonal antibodies reactive with the phytase protein.

Antibodies

Antibodies useful in the invention may be made using a mammal, in particular, a rabbit, chicken, mouse or a goat. The program for inoculation is not critical and may be any normally used for this purpose in the art. Such procedures are described, for example, in Antibodies A Laboratory Manual, Cold Spring Harbor Laboratory, 1988, pages 92-115.

The preferred antibodies for the detection of phytase are rabbit antibodies, chicken antibodies, and goat antibodies that are immunoaffinity purified against recombinant phytase produced in E. coli inclusion bodies. To detect and quantitate phytase, the antibodies are labelled, preferably, directly using labels which include enzymes, radioisotopes, and colored particles such as latex beads or colloidal gold. In another embodiment, the antibodies are indirectly labelled, for example, by reaction with labelled substances that bind to the antibody such as secondary antibodies, protein A or protein G.

Polyclonal Antibodies

In one embodiment, the antibodies are polyclonal antibodies. Methods for preparing polyclonal antibodies are known to the skilled artisan. Polyclonal antibodies can be raised in an animal, for example, by one or more injections of an immunizing agent and, if desired, an adjuvant. Typically, the immunizing agent and/or adjuvant will be injected in the mammal by multiple subcutaneous or intraperitoneal injections. The immunizing agent includes the feed enzyme or fusion protein thereof. For example, the agent is the phytase polypeptide or a fusion protein thereof. In another method, the immunizing agent is conjugated to a protein known to be immunogenic in the mammal being immunized. Examples of such immunogenic proteins include, but are not limited to, keyhole limpet hemocyanin, serum albumin, bovine thyroglobulin, and soybean trypsin inhibitor. Examples of adjuvants include Freund's complete adjuvant and MPL-TDM adjuvant (monophosphoryl Lipid A, synthetic trehalose dicorynomycolate). The immunization protocol can be selected by one skilled in the art without undue experimentation. The preferred antibodies are highly sensitive for the detection of feed enzymes such as but not limited to, phytase proteins, for example transgenic phytase proteins at relevant concentrations in bulk samples of commodity grain in the distribution channel. Preferably, the antibodies detect feed enzymes, such as phytase protein, at a high sensitivity of approximately 0.059 ng/ml. High sensitivity antibodies are useful for detection of low concentrations of feed enzymes, such as phytase proteins, in genetically engineered crop tissues, such as, but not limited to, leaf, stem, seed, stalk, root, and the like, or products derived from such crops, such as food fractions.

Monoclonal Antibodies

The anti-feed enzyme antibodies, such as anti-phytase antibodies are, alternatively, monoclonal antibodies. Monoclonal antibodies are prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent typically includes the desired polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells are cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically includes hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, 1987, pp. 51-63).

The culture medium in which the hybridoma cells are cultured is then be assayed for the presence of monoclonal antibodies directed against PRO. Preferably, the binding specificity of monoclonal antibodies produced by the hybridoma cells is determined by immuno-precipitation or by an in vitro binding assay, such as radio-immunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones are subcloned by limiting dilution procedures and grown by standard methods (Goding, supra). Suitable culture media for this purpose includes, for example, Dulbecco's Modified Eagle's Medium and RPMI-1640 medium. Alternatively, the hybridoma cells are grown in vivo as ascites in a mammal. The monoclonal antibodies secreted by the subclones are isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies are also be made by recombinant DNA methods, such as those described in U.S. Pat. No. 4,816,567. DNA encoding the monoclonal antibodies of the invention is readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA is placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also is modified, for example, by substituting the coding sequence for human heavy and light chain constant domains in place of the homologous murine sequences (U.S. Pat. No. 4,816,567; Morrison et al., supra), or by covalently joining to the immunoglobulin coding sequence to all or part of the coding sequence for a non-immunoglobulin polypeptide. Such a non-immunoglobulin polypeptide is substituted for the constant domains of an antibody of the invention, or is substituted for the variable domains of one antigen-combining site of an antibody of the invention to create a chimeric bivalent antibody.

In another embodiment, the antibodies are monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking. In vitro methods are also suitable for preparing monovalent antibodies. Digestion of antibodies to produce fragments thereof, particularly, Fab fragments, can be accomplished using routine techniques known in the art.

Other methods known in the art include the method of Keamey, et al., J. Immunol. 123: 1548-1558 (1979), which is incorporated by reference herein. Briefly, animals such as mice or rabbits are inoculated with the immunogen in adjuvant, and spleen cells are harvested and mixed with a myeloma cell line. The cells are induced to fuse by the addition of polyethylene glycol. Hybridomas are chemically selected by plating the cells in a selection medium containing hypoxanthine, aminopterin and thymidine (HAT). Hybridomas are subsequently screened for the ability to produce anti-phytase monoclonal antibodies. Hybridomas producing antibodies are cloned, expanded and stored frozen for future production.

In another embodiment, the antibody is labelled directly with a detectable label for identification and quantitation of a feed enzyme, in particular, a phytase protein. Labels for use in immunoassays are generally known to those skilled in the art and include, but are not limited to enzymes, radioisotopes and fluorescent, luminescent and chromogenic substances including colored particles such as colloidal gold and latex beads.

Alternatively, the antibodies are labelled indirectly by reaction with labelled substances that have an affinity for immunoglobulin, such as protein A or G or second antibodies. The antibodies are conjugated with a second substance and detected with a labelled third substance having an affinity for the second substance conjugated to the antibody. For example, the antibody is conjugated to biotin and the antibody-biotin conjugate detected using labeled avidin or strepavidin.

In another embodiment, the antibody is conjugated to a hapten and the antibody-hapten conjugate detected using labelled anti-hapten antibody. These and other methods of labelling antibodies and assay conjugates are well known to those skilled in the art.

Anti-feed enzyme, in particular anti-phytase, monoclonal and polyclonal antibodies having similar or superior sensitivity for feed enzyme proteins are produced by immunization of an animal with the feed enzyme, in particular the phytase protein described above, isolation of antibodies that react with the protein, and the collection and purification of the antibodies from a biological fluid such as blood in accordance with methods well known to those skilled in the art.

Immunoassay

The antibodies are collectively assembled in a kit with conventional immunoassay reagents for detection of the desired feed enzyme or protein using the immunoassay described below. The kit may optionally contain both monoclonal and polyclonal antibodies and a standard for determining the presence of the enzyme in a sample. The kit containing these reagents provides for simple, rapid, on site detection of the protein.

The antibodies described above are used as the basic reagents of a number of different immunoassays to determine the presence of the feed enzyme in a sample. The antibodies are employed in any type of immunoassay, whether qualitative or quantitiative.

In a typical quantitative sandwich assay, there are three basic parts. For example, in such as assay for a feed enzyme, such as phytase, the phytase protein in a genetically modified plant extract or feed extract, such as chicken feed, is captured onto the solid phase using a primary antibody. In one embodiment, the primary antibody is a rabbit anti-feed enzyme antibody. Then a “sandwich” is formed between the primary antibody, the feed enzyme protein, and the secondary antibody that has been added to the well. In one embodiment, the secondary antibody is a goat anti-feed enzyme antibody. After a wash step, where unbound secondary antibody has been removed, the bound secondary antibody is detected using a labelled antibody. In a particular embodiment, the detection antibody is an alkaline phosphatase-labelled donkey anti-goat antibody. Substrate for the detection enzyme, alkaline phosphatase, is added and color development is measured by reading the absorbance of each well. The standard curve uses a four-parameter curve fit to plot the concentrations versus the absorbance.

In one embodiment of the invention, the immunoassay for the detection of feed enzyme comprises the steps of:

a) preparing an extract of the sample;
b) incubating a portion of the extract with a primary anti-feed enzyme antibody which binds to the feed enzyme, the primary antibody being bound to a solid carrier, and a secondary anti-feed enzyme antibody which binds to the feed enzyme to create an antibody-polymer-antibody complex,
c) washing the antibody-polymer-antibody complex to remove unbound secondary antibody;
d) adding a detection antibody that immunogically reacts with the secondary antibody wherein the detection antibody is labelled; and
e) measuring the amount of bound or unbound labeled antibody to determine the concentration of the water treatment polymer in the fluid.

In pparticular embodiments, the feed enzyme is a phytase, xylanase, cellulase, glucanase, amylase, glucoamylase, and/or a protease protein. In a more preferred embodiment, the phytase is a thermostable phytase.

In another embodiment of the invention, the detectable label is an enzyme. In more preferred embodiments, the enzyme is alkaline phosphatase, peroxidase, or β-galactosidase.

In another embodiment, the enzyme produces an insoluble reaction product.

The invention also provides a kit for the detection and quantification by the immunoassay method comprising:
a) a means of extracting the feed enzyme from a sample;
b) a solid support comprising a primary anti-feed enzyme antibody bound to the solid support;
c) a secondary anti-feed enyzme antibody; and
d) a detection antibody capable of immunologically binding to the secondary antibody and wherein the detection antibody is labelled with a means of detection.

In a particular embodiment, the means of detection is an enzyme. In more preferred embodiments, the detection enzyme is alkaline phosphatase, peroxidase, or β-galactosidase.

In another embodiment, the enzyme produces a soluble or an insoluble reaction product.

In another embodiment, the kit further comprising a substrate for the enzyme.

Such immunoassays are also referred to enzyme-linked immunosorbent assays (ELISA).

The antibodies described above are also employed in a qualitative immunoassay for the detection of a feed enzyme, such as phytase. One such assay is commonly referred to as an immunostrip. An immunostrip is produced using membranes and filters through which a liquid sample is drawn by capillary action. The phytase in the sample reacts with the antibodies contained in the immunostrip as it moves the length of the strip. To detect phytase protein in chicken feed, the feed is washed with a buffer, separated from the solid material, and added to the immunostrip. As the liquid sample migrates to the opposite end of the immunostrip, the phytase reacts with the specific antibodies and is captured in a line that becomes visible. Detection of the signal on the test line indicates that phytase is in the sample.

In one embodiment the invention provides an immunoassay for the detection of a feed enzyme in a sample comprising the steps of:
a) preparing an extract of the sample in the presence of a primary antibody which immunologically recognizes the feed enzyme in the extract such that a primary antibody-feed enzyme complex is formed;
b) preparing a solid phase format having a significant measurement in three dimensions to form a substantial volume with a plurality of interstitial spaces by binding to it a desired secondary antibody capable of immunologically recognizing the feed enzyme and wherein the secondary antibody is conjugated to a means of detection and wherein the secondary antibody also immunologically recognizes the feed enzyme;
d) combining the extract of step (a) with the prepared format of step (b) whereby the extract is drawn through the interstitial spaces of the prepared solid phase format capturing the primary antibody-feed enzyme complex;
e) detecting the feed enzyme by the presence of said captured primary antibody-feed enzyme complex.

In other embodiments, the feed enzyme is a phytase, xylanase, cellulase, glucanase, amylase, glucoamylase, and/or a protease protein. In a preferred embodiment, the phytase is a thermostable phytase.

In other embodiments, the solid phase format is cellulose acetate, cellulose, nitrocellulose or nylon. In another embodiment, the solid phase format is composed of multiple stacked and contiguous layers wherein each layer is capable of capturing a different feed enzyme. In a preferred embodiment, the solid phase support further comprises a sample absorption pad of the solid phase format. In a more preferred embodiment, the immunoassay further comprises a strip comprising a labelled anti-feed enzyme antibody.

In a particular embodiment, the means of detection is colloidal gold.

A highly sensitive immunoassay employing the antibodies described above is provided. The assay is useful for the detection of genetically modified organisms that have been engineered to include a gene encoding a feed enzyme or protein, such as a phytase gene. The immunoassay is capable of detecting low concentrations of the protein in samples, such as animal feed and in genetically enhanced crop samples.

As described above, the antibodies used in the immunoassay are immuno-reactive with epitopes or a common epitope on the feed enzyme, in particular phytase protein, expressed by various micro-organisms and react minimally with other proteins that may be present in the sample, thus providing for an accurate determination of the presence of a genetically modified organism in a sample, such as a grain sample.

The immunoassay is useful for detecting the presence or amount of the desired feed enzyme protein, for example a phytase, in a variety of samples, including animal feed and agricultural samples such as plant material. The sample may be obtained from any source in which the desired protein is accessible to the antibody. For example, the sample may be any plant tissue or extract including root, stem, stalk, leaf, or seed or products derived from such crops, such as food fractions.

One or more of the antibodies described above are employed in any heterogeneous or homogeneous, sandwich or competitive immunoassay for the detection of a feed enzyme, in particular phytase protein, for example. Either the antibody is labelled with a detectable label or coupled to a solid phase. Methods for coupling antibodies to solid phases are well known to those skilled in the art. In accordance with the immunoassay method, the sample containing the feed enzyme is reacted with the antibody for a sufficient amount of time under conditions that promote the binding of antibody to phytase protein in the sample. It will be understood by those skilled in the art that the immunoassay reagents and sample may be reacted in different combinations and orders. A physical means is employed to separate reagents bound to the solid phase from unbound reagents such as filtration of particles, decantation of reaction solutions from coated tubes or wells, magnetic separation, capillary action, and other means known to those skilled in the art. It will also be understood that a separate washing of the solid phase may be included in the method.

The concentration of feed enzyme protein such as phytase in the sample is determined either by comparing the intensity of the color produced by the sample to a color card or by using a reflectometer.

The resulting reaction mixture, or combination of antibody and sample, is prepared in a solution that optimizes antibody-feed enzyme binding kinetics. An appropriate solution is an aqueous solution or buffer. The solution is preferably provided under conditions that will promote specific binding, minimize non-specific binding, solubilize the feed enzyme, stabilize and preserve reagent reactivity, and may contain buffers, detergents, solvents, salts, chelators, proteins, polymers, carbohydrates, sugars, and other substances known to those skilled in the art.

The reaction mixture solution is reacted for a sufficient amount of time to allow the antibody to react and bind to the feed enzyme to form an antibody-feed enzyme complex. The shortest amount of reaction time that results in binding is desired to minimize the time required to complete the assay. An appropriate reaction time period for an immunostrip test is less than or equal to 10 minutes or between approximately one minute and 10 minutes. A reaction time of less than five minutes is preferred. Most preferably, the reaction time is less than three minutes. By optimizing the reagents, binding may be substantially completed as the reagents are combined.

The reaction is performed at any temperature at which the reagents do not degrade or become inactivated. A temperature between approximately 18° C. and 30° C. is preferred, and most preferred reaction temperature is ambient or room temperature (approximately 22° C.).

A solid phase format such as an immunostrip is ideally suited for this immunoassay. Test strips are comprised of multiple porous components, membranes and filters, through which liquid sample is drawn by capillary action. The feed enzyme in the sample reacts with the test reagents contained within the test strip as it traverses the length of the strip. To detect protein in grain or seed, the grain is ground into a powder and the protein extracted from the powder with a liquid that is then separated from the solid material and assayed using the test. The liquid is applied to the immunostrip, and the feed enzyme migrates toward the distal end of the strip. As it migrates down the strip, the feed enzyme reacts with reagents applied to or immobilized on the strip causing a detectable signal product. Detection of the signal indicates the presence of the feed enzyme in the sample.

In one embodiment the solid phase format is cellulose acetate, cellulose, nitrocellulose or nylon. In a preferred embodiment, the solid phase format is nitrocellulose.

In another embodiment, the solid phase format comprises a sample absorption pad, a strip of nitrocellulose and a bottom pad comprising a labelled anti-feed enzyme antibody.

In yet another embodiment, the solid phase format is composed of multiple stacked and contiguous layers wherein each layer is capable of capturing a different feed enzyme.

Immunoassay Kit

An immunoassay kit for the detection of feed enzyme protein in a sample contains one or more of the antibodies described above. The kit may additionally contain equipment for obtaining the sample, a vessel for containing the reagents, a timing means, a buffer for diluting the sample, and a colorimeter, reflectometer, or standard against which a color change may be measured. The kit may include the reagents in the form of an immunostrip as described above.

In a preferred embodiment, the reagents, including the antibody are dry. Addition of aqueous sample to the vial or strip results in solubilization of the dry reagent, causing it to react.

The reagents, immunoassay methods, and kits described above will be further understood with reference to the following non-limiting examples. The examples below show typical experimental protocols and reagents that can be used in the detection of feed enzymes, in particular, phytase, in samples such as feed or other plant materials. Such examples are provided by way of illustration and not by way of limitation.

Numerous references cited above are all incorporated herein in their entireties.

### EXAMPLES

These methods and materials describe the general procedure for preparing the corn samples and the production of the monoclonal antibodies used in the examples described below.

Materials and Methods

Maize Sample: The corn extract was derived from either Hi II seed or A188 seed (non-transgenic) or genetically modified phytase seed. Five kernels were pulverized using a Kleco tissue grinder. The resulting corn flour was suspended in 5 mls distilled water to solubilize the proteins. The supernatant was tested in either the ELISA or with the immunostrips.

Production of Polyclonal Antibodies

For immunization: After the initial injection, the animal (rabbit or goat) is boosted after 28 days. Each subsequent boost thereafter is every 21 days. The animals are bled 10 days after each boost.

For chickens, the first boost is 7 days after the initial injection, followed by boosts every 28 days. The chickens are bled 10 days after each boost, and if a good antibody titer is detected, the eggs laid after the boost are collected.

The immunizing agent was the entire phytase protein purified from an E. coli expression system. With the first injection into the animal, the protein is emulsified in complete Freund's adjuvant. The boosts are in incomplete Freund's adjuvant. The animals we used to produce the polyclonal antibodies were rabbit, chicken, and goat.

Phytase (Nov9X) Purification:

Phytase (Nov9X) formulated with 10% sorbitol, 10% NaCl, and pH 4.2 was dialyzed overnight against 25 mM Tris-HCl, pH 8.0 at 4□C using SnakeSkin IOK MWCO dialysis tubing (Pierce, Rockford, Ill.). Following dialysis solid (NH₄)₂SO₄ was added to the phytase mixture, initially to 25% saturation, then to 50 and finally 75% saturation at 0° C. Upon the addition of (NH₄)₂SO₄ to 25% saturation the mixture was stirred for 30 minutes at 0° C., then centrifuged at 20,000 rpm for 20 minutes. To the decanted supernatant, (NH₄)₂SO₄ was added to 50% saturation while the pellet was resuspended in 25 mM Tris-HCl, pH 9.0. This procedure was carried out 3 times yielding Nov9X (NH₄)₂SO₄ pellets of 0-25%, 25-50%, and 50-75% saturation. SDS-PAGE analysis demonstrated the presence of Nov9X in the 50-75% fraction. This fraction was dialyzed against 25 mM Tris-HCl, pH 9.0 and prepared for column chromatography purification.

Crude Nov9X TAM from the 50-75% (NH₄)₂SO₄ fractionation was loaded onto a HiTrapQ anion exchange column (Amersham Biosciences, Piscataway, N.J.) using a flow rate of 5.0 mL/min. A linear gradient of 0-0.4 M NaCl in 25 mM Tris-HCl, pH 9.0 developed over 30 minutes was used to elute Nov9X. Absorbance measurements at 280 μm were used to follow the progress of the chromatography run. Following SDS-PAGE analysis the purest of the Nov9X containing fractions were pooled, concentrated with a Centricon Plus-20 centrifugal concentrator (Millipore, Bedford, Mass.), and loaded onto a 26/60 Sephacryl S100 size exclusion column (Amersham Biosciences, Piscataway, N.J.) run at 1 mL/min. The eluant buffer was 25 mM Tris-HCl, pH 9.0. Fractions containing pure Nov9X were pooled, concentrated, dialyzed against 25 mM Tris-HCl, pH 8.0, and used for the studies described below.

### Example 1

### Phytase ELISA

This example describes the detection and quantitative measurement of phytase enzyme in a corn sample using the ELISA immunological technique.

Procedure

The multiwell plates (Nunc, Maxisorp) were coated at 4° C. overnight with the rabbit anti-phytase antibody at a concentration of 2 μg/ml, diluted in borate buffered saline pH 8.5 (50 mM sodium borate/boric acid, 75 mM NaCl). The plates were washed five times with a Tris base buffer pH 8.0 (10 mM Tris containing 0.05% Tween-20 and 0.03% sodium azide) (wash buffer). Note: the same wash step was performed after each incubation period to remove unbound antibodies/samples. Plates were then blocked for 45 min. at room temperature with PBS/tween-20/BSA buffer pH 7.4 (1% bovine serum albumin, 0.05% Tween-20, 0.03% sodium azide, 150 mM NaCl in 100 mM sodium phosphate, pH 7.4 (diluent). Fifty microliters of each sample was added to the plate and incubated for 1.5 hr at room temperature. The goat anti-phytase antibody (diluted to 2 μg/ml in diluent) was then added to the plates and incubated for 1 hr at 37° C. The detection antibody (alkaline phosphatase-labelled donkey anti-goat antibody was diluted to 1 μg/ml in diluent) was added to the plates and incubated for 1 hr at 37° C. The substrate, paranitrophenylphosphate (pNPP) was added and allowed to develop for 30 min at room temperature. The absorbance was measured at 405 nm with 492 nm as a reference.

Assay Characteristics

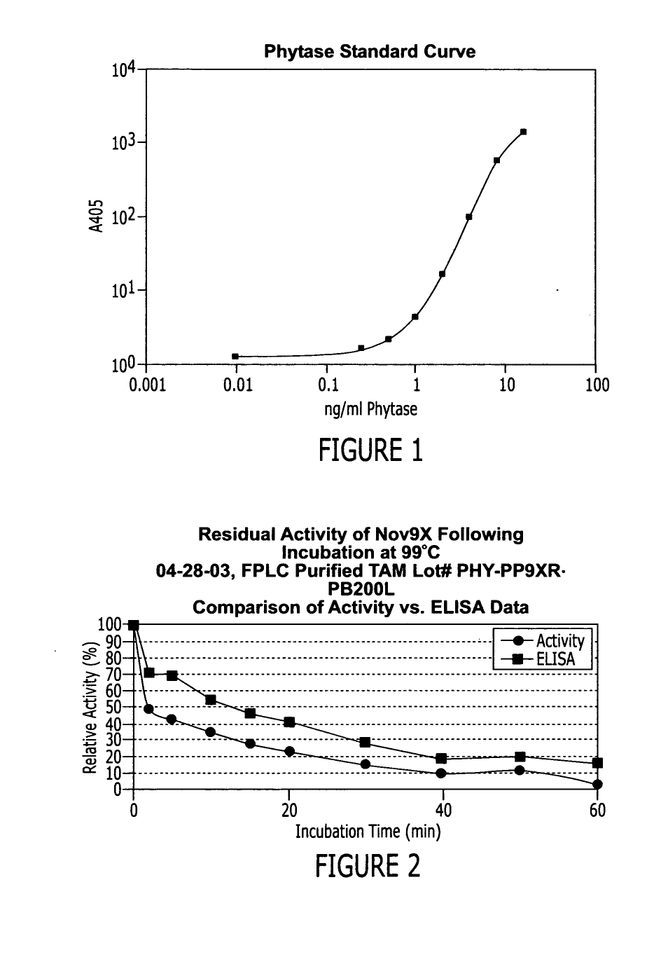

The phytase standard curve was a 4-parameter curve fit (see FIG. 1). The curve was plotted linear vs. log with a range from 0.04 to 16 ng/ml. To plot the 4-parameter standard curve on a log X axis, the 0 ng/ml standard must be entered into the analysis program at 0.01 ng/ml instead of 0 ng/ml. The analysis program used was WinSelect™ software for the Tecan Sunrise™ microplate reader, although any four-parameter curve-fitting program will work.

The minimum detectable dose (MDD) was the lowest level of phytase protein that was statistically distinguished from the zero standard. The minimum detectable dose was determined by analysis of 24 replicates of negative control corn seed extract at 1 mg/ml total protein. Two standard deviations of the zero standard mean O.D. (95% confidence limits) were added to the mean, and the dose of this total O.D. value was determined using a standard curve. The minimum detectable dose was 0.044 ng/ml.

Between-run precision was determined by assaying 4 different control samples in 21 different assays. The samples were purified phytase spiked into ELISA diluent. The results are set forth below in Table 1. The precision is good, less than 15%, for samples concentrations that are measured in the linear portion of the standard curve.

TABLE 1
Between-run Precision Test

| | Mean Phytase | Standard | % Coefficient |
|---|---|---|---|
| Sample | ng/ml | Deviation | of Variation |
| 1 | 9.65 | 2.27 | 23.5% |
| 2 | 2.99 | 0.38 | 12.8% |
| 3 | 0.94 | 0.11 | 12.0% |
| 4 | 0.39 | 0.10 | 25.6% |

Witn-run precision was determined by testing 20-24 replicates of the following samples. The samples were phytase spiked into ELISA diluent. The results are set forth in Table 2 below. All samples resulted in very good precision, indication good reproducibility within a single assay run.

TABLE 2
Within-run Precision Test

| | Mean Phytase | Standard | % Coefficient |
|---|---|---|---|
| Sample | ng/ml | Deviation | of Variation |
| 1 | 0.463 | 0.030 | 6.44% |
| 2 | 2.293 | 0.264 | 11.51% |
| 3 | 5.224 | 0.787 | 15.07% |

Four corn seed extracts were diluted with ELISA diluent in order to test the linearity of the assay. The corn extract was derived from either Hi II seed or A188 seed (non-transgenic) or genetically modified phytase seed. Five kernels were pulverized using a Kleco tissue grinder. The resulting corn flour was suspended in 5 mls distilled water to solubilize the proteins. The supernatant was tested in either the ELISA or with the strips. The percent recovery of phytase from the diluted samples was acceptable.

TABLE 3
Linearity of Assay Test

| | | | Measured | |
|---|---|---|---|---|
| | | Measured | Value X | Percent |
| Sample | Dilution | Phytase (ng/ml) | dilution faction | Recovery |
| A | 1/2500 | 12.76 | 31900 | 82% |
| | 1/5000 | 5.85 | 29250 | 75% |
| | 1/10,000 | 3.90 | 39000 | 100% |
| B | 1/2500 | 6.87 | 17175 | 52% |
| | 1/5000 | 6.90 | 34500 | 104% |
| | 1/10,000 | 3.33 | 33300 | 100% |
| C | 1/2500 | 3.58 | 8950 | 63% |
| | 1/5000 | 2.35 | 11750 | 83% |
| | 1/10,000 | 1.41 | 14100 | 100% |
| D | 1/2500 | 6.11 | 15275 | 72% |
| | 1/5000 | 3.54 | 17700 | 83% |
| | 1/10,000 | 2.13 | 21300 | 100% |

### Example 2

### Phytase Immunostrips

This example describes the use of Immunostrip assays to test the presence of phytase in a sample.

Procedure

Extracts of mashed chicken feed were prepared by adding feed to a 50 ml centrifuge tube up to the 15 ml designation. This amount of feed was added to one side of the mesh insert within the extraction bag. Extraction buffer (25 ml of 0.1 M borate pH 7.5 containing 0.5% Tween-20) was added and the buffer was gently pressed over the feed to ensure that all the feed was wet. The extract was incubated at room temperature for at least 10 min before applying 3-5 drops to the immunostrip for testing.

Immunostrip

Briefly, the lateral-flow immunostrip comprised a detection membrane of nitrocellulose (2.5×18 cm), supported on a plastic backing (Aristam™ brand plastic cassettes, Bethlehem, Pa.), in which a 1 mm line of specific rabbit (chicken antibodies can also be used) anti-phytase polyclonal antibody was sprayed. A reagent control line of donkey anti-goat antibody was sprayed in parallel above the first antibody line. The bottom end portion of the strip of nitrocellulose is over-layered with a piece of treated polyester strip. The polyester strip is first treated with a solution B (0.5% BSA, 0.5% polyvinylalcohol and 0.1% Triton X-100; 50 mM phosphate buffer pH 7.4) and the colloidal gold conjugated goat anti-phytase antibody. The polyester strip is allowed to dry. The polyester strip is then overlayered with a sample application pad of cotton. The sample application pad was also pretreated with a solution C (0.1 ! Triton X-100 and 0.1 M borate buffer pH 8.5) and allowed to dry. Flanking the other end or top end of the nitrocellulose strip is another cotton pad to absorb the solution from the sample after it passes over the test antibody and control antibody areas on the nitrocellulose. This completed card was then cut into 4 mm test strips to fit into a plastic cassette with an oval sample application well positioned above the sample pad and a rectangular detection window positioned above the detection area of the nitrocellulose membrane.

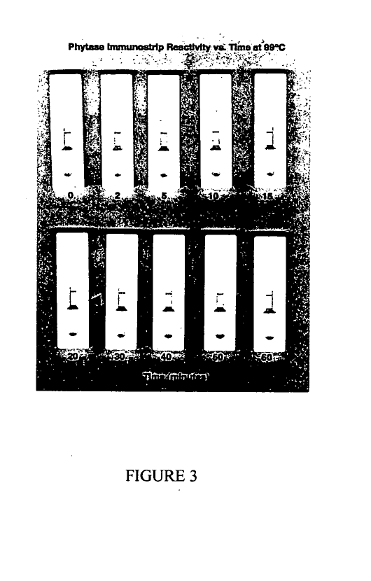

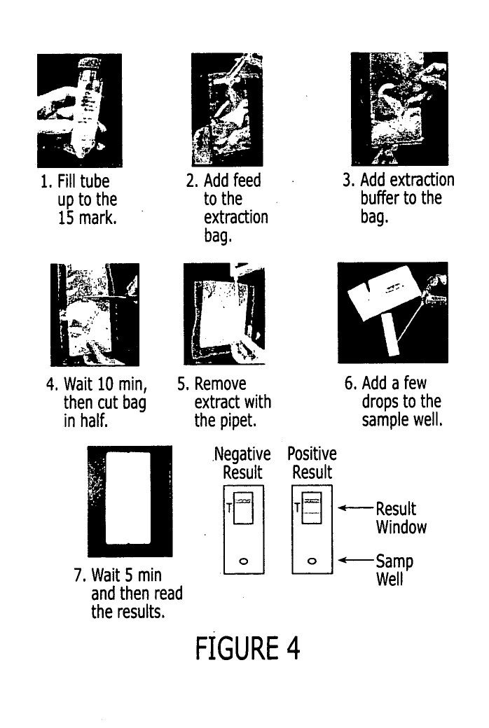

The assay was performed by adding 150 μl (3-5 drops) of extract to the sample well. After waiting approximately 5-10 minutes, the results appeared in the result window. If phytase was present in the sample, a double red line appeared in the result window. The lower line indicates the presence of phytase while the upper line is the control line demonstrating a properly working device. If phytase is absent, only one single red control line appears in the result window. See FIGS. 3 and 4 for sample immunostrips. FIG. 3 shows the detection of the presence of phytase. The detection of phytase decreases after 20 minutes as indicated by the arrow, because that is when the phystase is starting to lose activity.

Detailed Preparation of the Immunostrip

### Phytase 2^{nd} Generation Strips—Coating of Membrane

Materials

1. Cards, 2.25 in.×180 mm, with AE100 membrane
2. Chicken anti-phytase IAP at 1.0 mg/ml in PBS
3. Jackson donkey anti-goat antibody at 0.15 mg/ml in PBS
4. Pierce RBS detergent
   Procedure

Coating the Test Line:
1. Set the Camag™ sprayer volume to 18 (1 μl/cm) by pressing vol, 18, enter, enter.
2. Set the track by pressing track, 1, enter, enter.
3. Place the card on the platform. The portion of the card with the 2 paper pieces is placed closest to the front of the instrument. Secure the card with the magnets.
4. Fill the syringe with 1.0 mg/ml chicken anti-phytase IAP.
5. Set the spray head at 30 mm.
6. Turn on the gas supply and begin the spray by pressing gas, calc, run. Watch the spray pattern closely for consistency and accuracy.
7. Repeat steps 3-6 for each additional card.
8. Remove syringe and wash 5 times with Pierce RBS detergent (20 μl concentrate/ml dH₂O), then wash 10 times with dH₂O.
   Coating the Control Line:
9. Set the Camag volume to 18 (1 μl/cm) by pressing vol, 18, enter, enter.
10. Set the track by pressing track, 1, enter, enter.
11. Fill the syringe with 0.15 mg/ml donkey anti-goat.
12. Set the spray head at 36 mm.
13. Turn on the gas supply and begin the spray by pressing gas, calc, run. Watch the spray pattern closely for consistency and accuracy.
14. Repeat steps 11-13 for each additional card.
15. Remove syringe and wash 5 times with Pierce RBS detergent (20 μl concentrate/ml dH₂O), then wash 10 times with dH₂O.
16. Dry cards at 33° C. overnight, then transfer to room temperature.
17. Store desiccated at RT.

### Phytase Strips—Coating the Conjugate onto Polyester

Materials

1. Gold conjugated goat anti-NOV9X, OD=50
2. Polyester sheets, 2033 grade, treated with solution B
3. Sucrose
4. Trehalose
5. Pierce RBS detergent
   Procedure
18. Dilute gold conjugate to OD=50 using gold diluent.
19. Add 20% sucrose and 5% trehalose to the gold conjugate to stabilize (0.2 g sucrose and 50 mg trehalose per 1 ml gold conjugate). Mix until completely dissolved.
20. Set the Camag™ volume to 27 (1.5 μl/cm) by pressing vol, 27, enter, enter.
21. Set the track by pressing track, 1, enter, enter.
22. Place the polyester sheet on the platform and secure with the magnets.
23. Fill the syringe with stabilized gold conjugated goat anti-NOV9X (OD=50).
24. Set the spray head at 15 mm.
25. Turn on the gas supply and begin the spray by pressing gas, calc, run. Watch the spray pattern closely for consistency and accuracy.
26. Move the spray head 9 mm (setting will be 24 mm).
27. Turn on the gas supply and begin the spray by pressing gas, calc, run.
28. Continue spraying the conjugate, moving 9 mm for each run, until the entire polyester sheet is filled. Eight lines of conjugate will fill a sheet.
29. Dry the sheet at 37° C. for 1 hr.
30. Cut into ¼″ strips such that the line of gold conjugate runs along the top of each strip.
31. Store desiccated at RT.
   Cleaning of Instrument
Remove syringe and wash 5 times with Pierce RBS detergent (20 μl concentrate/ml
dH₂O), then wash 10 times with dH₂O.
Clean instrument platform with dH₂O.

### Phytase Strips—Assembly

Materials
6. Cards coated with chicken anti-phytase LAP antibody at 1.0 mg/ml and 1 μl/cm.
7. ⅝″×180 mm strips of #40 absorbant paper (top pad)
8. ¾″×180 mm strips of #903 paper treated with solution C, pH 8.6 (bottom pad).
9. ¼″×180 mm strips of sprayed gold conjugate (goat anti-NOV9X, OD=50 at 1.5 μl/cm).
10. gloves
   Procedure
   Note: Assemble strips under conditions of less than 40% humidity. Wear gloves to apply all components.
1. Remove the two liners from the glue strips at the bottom of the card.
2. Position the gold strip with the line of gold conjugate along the top and overlapping the membrane by 1-1.5 mm.
3. Place the bottom pad along the bottom edge of the card, taking care to leave the gold strip exposed.
4. Remove the liner form the glue strips along the top of the card. Place the top pad along the top of the card overlapping the membrane by 1-1.5 mm.
5. Store the finished cards desiccated at RT until ready for cutting into strips.
6. Cut strips into 4 mm lengths. One card will yield ˜40 strips. Store strips desiccated at RT.

### Example 3

### Detection of Enzymatically Active Phytase

Procedure

Pichia produced purified phytase was inactivated by heating to 99° C. for up to 60 minutes. The phytase was then tested for enzyme activity and compared to reactivity in the phytase ELISA (FIG. 2) and reactivity with the phytase immunostrips (FIG. 3).

ELISA comparison: FIG. 2 shows a graph of the Residual activity of Nov9X following incubation at 99° C. 04-28-03, FPLC purified TAM Lot # PHY—PP9XR-PB200L Comparison of Activity vs. ELISA Data This demonstrates that the ELISA assay and the immunostrips appear to detect active phytase only. Phytase inactivated by heating is not detected in either assay.

### Example 4

### Phytase Immunoassay Kit

This diagnostic test (see FIG. 4) was designed for the rapid (10 min) detection of phytase in feed. The kit contains all reagents and equipment needed to perform the test. The kit can be stored at ambient temperatures not exceeding 100° F. (38° C.). The tests are packaged in a sealed moisture-proof foil bag with a silica gel desiccant capable of absorbing some moisture. Keep the test in its package until prior to its use. Avoid placing the test in a damp place.

Assay Procedure

1. Fill the large tube with feed up to the 15 mark. Add this amount of feed to one side of the mesh insert within the extraction bag.
2. Remove one plastic container of extraction buffer (25 ml) from kit and pour into the extraction bag.
3. Close bag and gently move the buffer over the feed to ensure that all the feed is wet. Wait at least 10 minutes.
4. Remove a Field Test from the foil bag and place on a flat dry surface. Check the desiccant. It should be blue. If it is pink, the tests are no longer valid and should be discarded.
5. Using the transfer pipet, transfer 3-5 drops of the feed extract to fill the sample well of the field test.
6. Wait approximately 5 minutes for the results to appear in the window above the sample well.
   Results

If phytase is present in the sample, a double red line appears in the result window of the field test. The lower line indicates the presence of phytase, while the upper line is the control line signaling a properly working device. The test line will not be as strong as the control line. Any reaction seen at the test line is considered positive.

If no phytase is present, only one single red control line appears in the result window.

### Example 5

### Detection of Phytase in Pelleted Feed

This example demonstrates the use of the immunostrip assays to detect phytase in pelleted animal feed.

The methods and reagents are described as above in Example 4, with the exception that the pelleted animal feed is crushed to a grainy or powdery consistency with any menchanical device, and that the extraction buffer was 5% methanol with 0.5% Tween-20 in water instead of the borate buffer. Also, the anti-phytase antibody was from chicken instead of rabbit. The results are set forth below in Table 4. Table 4 shows that Quantum® phytase was detectable in both mashed (before pelleting) and pelleted diets using both ELISA and immunostrip assays. Table 5 shows that Quantum phytase is detectable in the starter diets (before pelleting) and the crumber diets (pelleted diets) with both the immunostrip and the ELISA. Activity was also confirmed with the enzyme assay.

TABLE 4
Detection of Phytase in Pelleted Feed

| | Average | Phytase Level | |
|---|---|---|---|
| Diet | ng/ml | Added | Type |
| RA0309 Starter Diet 1 | 0 | 0 | mash |
| RA0309 Starter Diet 9 | 0 | 0 | pellet |
| RA0309 Starter Diet 4 | 0 | 0 | mash |
| RA0309 Starter Diet 15 | 0 | 0 | pellet |
| RA0309 Starter Diet 11 | 22.3375 | 285 | mash |
| RA0309 Starter Diet 19 | 24.9875 | 285 | mash |
| RA0309 Starter Diet 2 | 8.8425 | 285 | pellet |
| RA0309 Starter Diet 23 | 10.93 | 285 | pellet |
| RA0309 Starter Diet 6 | 27.495 | 566 | mash |
| RA0309 Starter Diet 17 | 46.25 | 566 | mash |
| RA0309 Starter Diet 13 | 19.76 | 566 | pellet |
| RA0309 Starter Diet 21 | 24.425 | 566 | pellet |
| RA0309 Starter Diet 3 | 58.19 | 1133 | mash |
| RA0309 Starter Diet 14 | 69.0275 | 1133 | mash |
| RA0309 Starter Diet 7 | 20.7225 | 1133 | pellet |
| RA0309 Starter Diet 22 | 32.825 | 1133 | pellet |
| RA0309 Starter Diet 12 | 153.62 | 2832 | mash |
| RA0309 Starter Diet 24 | 173.7425 | 2832 | mash |
| RA0309 Starter Diet 10 | 104.2125 | 2832 | pellet |
| RA0309 Starter Diet 28 | 100.6525 | 2832 | pellet |
| RA0309 Starter Diet 5 | 0 | 305 | Ronozyme |
| RA0309 Starter Diet 26 | 0 | 605 | Ronozyme |

TABLE 5
Phytase Activity and ELISA quantitation of
Phytase in Starter and Crumbled Diets

| | Extractable Activity | ELISA Result |
|---|---|---|
| | Average (FTU/kg) | ng/ml |
| Starter Diets | | |
| T1 | 37.7 | 0.0 |
| T2 | 301.1 | 4.9 |
| T3 | 426.3 | 16.7 |
| T4 | 74.8 | 0.0 |
| T5 | 209.8 | 7.5 |
| T6 | 449.3 | 17.3 |
| T7 | 58.0 | 0.0 |
| T8 | 152.7 | 4.0 |
| T9 | 806.6 | 13.3 |
| T10 | 436.5 | 18.4 |
| Crumbler Diets | | |
| T1 | 50.6 | 0.0 |
| T2 | 142.2 | 4.5 |
| T3 | 353.7 | 11.4 |
| T4 | 68.7 | 0.0 |
| T5 | 167.7 | 12.7 |
| T6 | 237.8 | 9.1 |
| T7 | 50.4 | 0.9 |
| T8 | 234.4 | 8.8 |
| T9 | 301.7 | 13.6 |
| T10 | 711.0 | 22.5 |

Modifications of the present reagents, methods and kits for detecting feed enzyme proteins, in particular phytase, will be obvious to those skilled in the art from the foregoing detailed description.

While the present invention has been described with reference to specific embodiments thereof, it will be appreciated that numerous variations, modifications, and further embodiments are possible, and accordingly, all such variations, modifications and embodiments are to be regarded as being within the scope of the present invention. Numerous patents, applications and references are discussed or cited within this specification, and all are incorporated by reference in their entireties.

1. An immunoassay for the detection of a feed enzyme in a sample comprising the steps of:
a) preparing an extract of the sample in the presence of a primary antibody which immunologically recognizes the feed enzyme in the extract such that a primary antibody-feed enzyme complex is formed;
b) preparing a solid phase format having a significant measurement in three dimensions to form a substantial volume with a plurality of interstitial spaces by binding to it a desired secondary antibody capable of immunologically recognizing the feed enzyme and wherein the secondary antibody is conjugated to a means of detection and wherein the secondary antibody also immunologically recognizes the feed enzyme;
d) combining the extract of step (a) with the prepared format of step (b) whereby the extract is drawn through the interstitial spaces of the prepared solid phase format capturing the primary antibody-feed enzyme complex;
e) detecting the feed enzyme by the presence of said captured primary antibody-feed enzyme complex.

2. The immunoassay of claim 1 wherein the feed enzyme is a phytase, xylanase, cellulase, glucanase, amylase, glucoamylase, and/or a protease protein.

3. The immunoassay of claim 2 wherein the phytase is a thermostable phytase.

4. The immunoassay of claim 1 wherein the solid phase format is cellulose acetate, cellulose, nitrocellulose or nylon.

5. The immunoassay of claim 4, wherein the solid phase format is composed of multiple stacked and contiguous layers wherein each layer is capable of capturing a different feed enzyme.

6. The immunoassay of claim 4, further comprising a sample absorption pad of the solid phase format.

7. The immunoassay of claim 6 further comprising a strip comprising a labelled anti-feed enzyme antibody.

8. The immunoassay of claim 1 wherein the means of detection is colloidal gold.

9. A kit for detection by the immunoassay of claim 1 comprising:
a) a means of extraction of the feed enzyme from a sample; and
b) a solid phase format comprising a primary anti-feed enzyme antibody and having a significant measurement in three dimensions to form a substantial volume with a plurality of interstitial spaces by binding to it a desired secondary antibody capable of immunologically recognizing the feed enzyme and wherein the secondary antibody is conjugated to a means of detection and wherein the secondary antibody also immunologically recognizes the feed enzyme.

10. The kit of claim 9 further comprising a vessel containing a buffer.

11. The kit of claim 10 further comprising a means of dispensing the sample onto the solid phase format.

12. An immunoassay for the detection and quantification of a feed enzyme comprising the steps of:
a) preparing an extract of the sample;
b) incubating a portion of the extract with a primary anti-feed enzyme antibody which binds to the feed enzyme, the primary antibody being bound to a solid carrier, and a secondary anti-feed enzyme antibody which binds to the feed enzyme to create an antibody-polymer-antibody complex,
c) washing the antibody-polymer-antibody complex to remove unbound secondary antibody;
d) adding a detection antibody that immunogically reacts with the secondary antibody wherein the detection antibody is labelled; and
e) measuring the amount of bound or unbound labeled antibody to determine the concentration of the water treatment polymer in the fluid.

13. The immunoassay of claim 12 wherein the feed enzyme is a phytase, xylanase, cellulase, glucanase, amylase, glucoamylase, and/or a protease protein.

14. The immunoassay of claim 13 wherein the phytase is a thermostable phytase.

15. The immunoassay of claim 12 wherein the detectable label is an enzyme.

16. The immunoassay of claim 15 wherein the enzyme is alkaline phosphatase, peroxidase, or β-galactosidase.

17. The immunoassay of claim 16, wherein the enzyme produces an insoluble reaction product.

18. A kit for the detection and quantification by the immunoassay of claim 12 comprising:
a) a means of extracting the feed enzyme from a sample;
b) a solid support comprising a primary anti-feed enzyme antibody bound to the solid support;
c) a secondary anti-feed enyzme antibody; and
d) a detection antibody capable of immunologically binding to the secondary antibody and wherein the detection antibody is labelled with a means of detection.

19. The kit of claim 18 wherein the means of detection is an enzyme.

20. The kit of claim 19 wherein the detection enzyme is alkaline phosphatase, peroxidase, or β-galactosidase.

21. The kit of claim 20 wherein the enzyme produces a soluble or an insoluble reaction product.

22. The kit of claim 21 further comprising a substrate for the enzyme.

23. An antibody that immunologically recognizes phytase

24. The antibody of claim 23, wherein the antibody is a polyclonal antibody.

25. The antibody of claim 23, wherein the antibody is a monoclonal antibody.
